(19) United States
(12) Patent Application Publication
(10) Pub. No.: US 2004/0241665 A1
(43) Pub. Date: Dec. 2, 2004

(54) Methods and devices for identifying a fluid on a substrate surface

(76) Inventors: Jay K. Bass, Mountain View, CA (US); Michelle M. Maranowski, San Jose, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/452,685

(22) Filed: May 30, 2003

(51) Int. Cl. C12Q 1/68; G01N 33/53; G06F 19/00; G01N 33/48; G01N 33/50; C12M 1/34

(52) U.S. Cl. 435/6; 435/7.1; 435/287.2; 702/20

(57) ABSTRACT

Methods and devices for identifying a fluid on a substrate surface are provided. In accordance with the subject invention, fluid is deposited onto a substrate surface from a fluid deposition device, e.g., a pulse-jet fluid deposition device, to produce a spot of the fluid on the substrate surface. The fluid is identified by evaluating at least one physical characteristic of the deposited spot. Also provided are computer-readable mediums that include a program for controlling an apparatus, such as a fluid deposition device, e.g., a pulse-jet fluid deposition device, to measure at least one physical characteristic of a spot deposited on a substrate surface from a fluid deposition device and evaluate the measurement to identify the deposited fluid. Kits for use in practicing the subject methods are also provided.

### FIELD OF THE INVENTION

The field of this invention is fluid identification, particularly fluid employed for in situ protocols for the synthesis of arrays.

### BACKGROUND OF THE INVENTION

Array assays between surface bound binding agents or probes and target molecules in solution may be used to detect the presence of particular analytes or biopolymers in a solution. The surface-bound probes may be oligonucleotides, peptides, polypeptides, proteins, antibodies or other molecules capable of binding with target biomolecules in the solution. Such binding interactions are the basis for many of the methods and devices used in a variety of different fields, e.g., genomics (in sequencing by hybridization, SNP detection, differential gene expression analysis, identification of novel genes, gene mapping, finger printing, etc.) and proteomics.

One typical array assay method involves biopolymeric probes immobilized in an array on a substrate such as a glass substrate or the like. A solution suspected of containing an analyte or target molecule(s) (“target(s)”) that binds with the attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern, i.e., a binding complex pattern, on the surface of the substrate which is detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

There are two main ways of producing polymeric arrays in which immobilized polymers are covalently attached to the substrate surface: via in situ synthesis in which the polymers are grown on the surface of the substrate in a step-wise fashion and via deposition of the full polymer, e.g., a pre-synthesized nucleic acid/polypeptide, cDNA fragment, etc., onto the surface of the substrate.

The in situ synthesis protocols include those described in U.S. Pat. No. 5,449,754 for synthesizing peptide arrays, as well as WO 98/41531 and the references cited therein for synthesizing polynucleotides (specifically DNA) using phosphoramidite or other chemistry. Such in situ synthesis methods may be generally regarded as iterating the sequence of depositing droplets of (a) a protected monomer onto predetermined locations on a substrate to link with either a suitably activated substrate surface or with a previously deposited deprotected monomer; (b) deprotecting the deposited monomer so that it can react with a subsequently deposited protected monomer; and (c) depositing another protected monomer for linking. Different monomers may be deposited at different regions on the substrate during any one cycle so that the different regions of a completed array will carry the different biopolymer sequences as desired in the completed array. One or more further steps may be required in each iteration, such as activation, oxidation, washing steps, etc.

For example, for the in situ synthesis of nucleic acid arrays, conventional phosphoramidite synthesis protocols are typically used as noted above. In phosphoramidite synthesis protocols, the 3′-hydroxyl group of an initial 5′-protected nucleoside is first covalently attached a substrate surface. Synthesis of the nucleic acid then proceeds by deprotection of the 5′-hydroxyl group of the attached nucleoside, followed by coupling of an incoming nucleoside-3′-phosphoramidite to the deprotected 5′ hydroxyl group (5′-OH). The resulting phosphite triester is finally oxidized to a phosphotriester to complete the internucleotide bond. The steps of deprotection, coupling and oxidation are repeated until a nucleic acid of the desired length and sequence is obtained.

Regardless of the type of array, i.e., whether the array is a nucleic acid array, peptides, etc., oftentimes in situ synthesis is carried-out by way of highly automated methods that employ in situ array synthesis devices such as pulse-jet fluid deposition devices in which thermal or piezo pulse jet devices analogous to inkjet printing devices are employed to deposit fluids of biopolymeric precursor molecules, i.e., monomers, onto a substrate surface. In this manner, a series of droplets, e.g., each containing one particular type of reactive deoxynucleoside phosphoramidite, may be sequentially applied to each discrete area or “feature”, sometimes referred to as a “spot”, of the array by a pulse-jet printhead. These automated deposition devices are typically configured to have one or more reservoirs, each containing a specific reagent such as a particular monomer, activator, etc., in communication with one or more printheads of the device. The reagents of the reservoirs are thus deposited onto a substrate surface via the printheads of the device. U.S. Patents disclosing thermal and/or piezo pulse jet deposition of biopolymer containing fluids onto a substrate include: U.S. Pat. Nos. 6,242,266; 6,232,072; 6,180,351; 6,171,797 and 6,323,0434, the disclosures of which are herein incorporated by reference.

It will be apparent that the in situ synthesis process must be a precise process such that the correct reagents must be employed in the correct order at precise positions on a substrate to synthesize a specific biopolymer. Typically, prior to being operatively associated with one or more appropriate printheads, the reagents employed in an in situ synthesis protocol, e.g., phosphoramidite and tetrazole reagents, may be prepared by a researcher, placed into a reservoir, labeled and then installed on a pulse-jet fluid deposition device. However, there is no full-proof way to verify the proper connection or rather arrangement of reagents, i.e., that the reservoirs are connected to the appropriate printheads and that the reagent have not been inadvertently mixed-up. Typically, such verification is accomplished manually by operators; however this method is prone to human error.

Accordingly, there continues to be an interest in the development of new methods and devices to verify the reagents employed by a fluid deposition device such as a pulse-jet fluid deposition device or other suitable fluid deposition device. Of particular interest is the development of such methods and devices that are easy to use, cost effective, effective at identifying a fluid deposited onto a substrate surface by a fluid deposition device and which may be partially or completely automated.

### SUMMARY OF THE INVENTION

Methods and devices for identifying a fluid on a substrate surface are provided. In accordance with the subject invention, fluid is deposited onto a substrate surface from a fluid deposition device such as a pulse-jet fluid deposition device or other suitable fluid deposition device to produce a spot of the fluid on the substrate surface. The fluid is identified by evaluating at least one physical characteristic of the deposited spot. Also provided are computer-readable mediums that include a program for controlling an apparatus, such as a fluid deposition device, e.g., a pulse-jet fluid deposition device and the like, to measure at least one physical characteristic of a spot deposited on a substrate surface from a fluid deposition device and evaluate the measurement to identify the deposited fluid. Kits for use in practicing the subject methods are also provided.

### BRIEF DESCRIPTIONS OF THE DRAWINGS

FIG. 1 illustrates an exemplary embodiment of the subject invention wherein a fluid is being deposited from a pulse-jet fluid deposition device onto a substrate surface.

FIG. 2 shows a spot of the fluid of FIG. 1 deposited onto the substrate surface.

FIG. 3 shows an exemplary embodiment of a fluid spot deposited onto a substrate surface according to the subject methods wherein one or more axes of the spot may be evaluated to determine the identity of the spot.

FIG. 4 shows an exemplary embodiment of a plurality of fluid spots deposited onto a substrate surface according to the subject methods.

FIG. 5 shows an exemplary embodiment of a fluid spot deposited onto a substrate surface according to the subject methods wherein the shape and/or size of the spot may be determined by the amount of substrate surface area contacted by the spot, e.g., by employing a grid.

FIGS. 6-9 show the experimental results of employing the subject methods of identify six fluids.

### DEFINITIONS

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in hybridization reactions, i.e., cooperative interactions through Pi electrons stacking and hydrogen bonds, such as Watson-Crick base pairing interactions, Wobble interactions, etc.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The term “monomer” as used herein refers to a chemical entity that can be covalently linked to one or more other such entities to form an oligomer. Examples of “monomer” include nucleotides, nucleosides, amino acids, saccharides, peptides, and the like. In general, the monomers used in conjunction with the present invention have first and second sites (e.g., C-termini and N-termini, or 5′ and 3′ sites) suitable for binding to other like monomers by means of standard chemical reactions (e.g., condensation, nucleophilic displacement of a leaving group, or the like), and a diverse element which distinguishes a particular monomer from a different monomer of the same type (e.g., an amino acid side chain, a nucleotide base, etc.). In certain embodiments, an initial monomer, such as a substrate-bound monomer, may be used as a building-block in a multi-step synthesis procedure to form a complete polymer or ligand, such as in the synthesis of oligonucleotides, oligopeptides, and the like. Monomers are usually, though not always, present in a liquid, typically in solution, where such may be referred to as a “fluid monomer”. In describing the subject invention, a monomer includes a monomer alone or with a suitable medium such as a fluid medium or the like. As such, a monomer and a fluid monomer may be used interchangeably herein.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

“Optional” or “optionally” means that the subsequently described circumstance may or may not occur, so that the description includes instances where the circumstance occurs and instances where it does not.

An “array,” includes any one or two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a surface of a substrate. Depending upon the use, any or all of the arrays may be the same or different from one another and each may contain multiple spots or features. A typical array may contain more than ten, more than one hundred, more than one thousand, more than ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents, but may or may not be present when other fabrication processes are employed. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (in many embodiments rectangular) area in which the array spots or features of interest, as defined above, are found. The scan region is that portion of the total area illuminated from which the resulting fluorescence is detected and recorded. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

A “biopolymer” is a polymer of one or more types of repeating units. Biopolymers are typically found in biological systems (although they may be made synthetically) and particularly include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions.

“Remote location,” means a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information references transmitting the data representing that information as electrical signals over a suitable communication channel (e.g., a private or public network).

“Forwarding” an item refers to any means of getting that item from one location to the next, whether by physically transporting that item or otherwise (where that is possible) and includes, at least in the case of data, physically transporting a medium carrying the data or communicating the data.

The terms “reporter,” “label” “detectable reporter” and “detectable label” refer to a molecule capable of generating a measurable signal, including, but not limited to, fluorescers, and the like. The term “fluorescer” refers to a substance or a portion thereof which is capable of exhibiting fluorescence in the detectable range when excited at the appropriate wavelength.

A “computer-based system” refers to the hardware means, software means, and data storage means used to perform certain functions and or analyze the information of the present invention. The minimum hardware of the computer-based systems of the present invention may include a central processing unit (CPU), input means, output means, and data storage means. A skilled artisan can readily appreciate that any one if the currently available computer-based systems are suitable for use in the present invention. The data storage means may include any manufacture including a recording of information relating to the subject invention, or memory access means that can access such a manufacture.

To “record” data, programming or other information on a computer-readable medium refers to a process for storing information, using any such methods as are known in the art. Any convenient storage structure may be chosen, based on the means to access the stored information. A variety of data processor programs and formats may be used for data storage, e.g., word processing text file, databases format, etc.

A “processor” references any hardware and/or software combination that will perform the functions required of it. For example, a processor herein may be a programmable digital microprocessor such as available in the form of an electronic controller, mainframe, server or personal computer )desktop or portable). Suitable programming may be communicated from a remote location to the processor, or previously saved in a computer program product (such as a portable or fixed computer-readable storage medium, whether magnetic, optical or solid state device based). For example, a magnetic or optical disk may carry the programming, and can be read by a suitable disk reader communicating with a respective processor at its corresponding station.

“Activator” refers to any suitable chemical and/or physical entity that is employed to make-possible, assist, enhance or increase in the joining or linking of a monomer to another chemical entity such as one or more other monomers or a reactive functional group such as a free hydroxy functional group present on a substrate surface, etc. For example, an activator may protonate a monomer so that it may be joined to another monomer or to a free functional group. For example, activators may be employed in phosphoramidite chemistry where they used in the joining of a deoxynucleoside phosphoramidite to a functional group present on a substrate surface or to another deoxynucleoside phosphoramidite. In producing nucleic acids on a substrate surface using phosphoramidite chemistry, one of the first steps in such a protocol involves attaching a first monomer to the substrate surface. Accordingly, a solution containing a protected deoxynucleoside phosphoramidite and an activator, such as tetrazole, benzoimidazolium triflate (“BZT”), S-ethyl tetrazole, and dicyanoimidazole, is applied to the surface of a substrate that has been chemically prepared to present reactive functional groups such as, for example, free hydroxyl groups. The activators tetrazole, BZT, S-ethyl tetrazole, and dicyanoimidazole are acids that protonate the amine nitrogen of the phosphoramidite group of the deoxynucleoside phosphoramidite. A free hydroxyl group on the surface of the substrate displaces the protonated secondary amine group of the phosphoramidite group by nucleophilic substitution and results in the protected deoxynucleoside covalently bound to the substrate via a phosphite triester group. An analogous methodology using an activator may be employed to link two deoxynucleoside phosphoramidites together such as a deoxynucleoside phosphoramidite to a substrate bound nucleotide. For example, a protected deoxynucleoside phosphoramidite in solution with an activator is applied to the substrate-bound nucleotide and reacts with the 5′ hydroxyl of the nucleotide to covalently link the protected deoxynucleoside to the 5′ end of the nucleotide via a phosphite triester group. In accordance with the subject invention, suitable “activators” include, but are not limited to, tetrazole and tetrazole derivatives such as S-ethyl tetrazole, dicyanoimidazole (“DCI”), benzimidazolium triflate (“BZT”), and the like. Activators are usually, though not always, present in a liquid, typically in solution, where such may be referred to as a “fluid activator”. In describing the subject invention, an activator includes an activator alone or with a suitable medium such as a fluid medium or the like. As such, an activator and a fluid activator may be used interchangeably herein.

### DETAILED DESCRIPTION OF THE INVENTION

Methods and devices for identifying a fluid on a substrate surface are provided. In accordance with the subject invention, fluid is deposited onto a substrate surface from a fluid deposition device such as a pulse-jet fluid deposition device or other suitable fluid deposition device to produce a spot of the fluid on the substrate surface. The fluid is identified by evaluating at least one physical characteristic of the deposited spot. Also provided are computer-readable mediums that include a program for controlling an apparatus, such as a pulse-jet fluid deposition device or other suitable fluid deposition device, to measure at least one physical characteristic of a spot deposited on a substrate surface from a fluid deposition device such as a pulse-jet fluid deposition device and the like and evaluate the measurement to identify the deposited fluid. Kits for use in practicing the subject methods are also provided.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The figures shown herein are not necessarily drawn to scale, with some components and features being exaggerated for clarity.

In further describing the subject invention in greater detail, the subject methods are described first, followed by a review of the devices of the subject invention. Also provided are discussions of representative applications in which the subject methods and devices may find use. Finally, kits for use in practicing the subject methods are described.

Methods of Identifying a Fluid Present on a Substrate Surface

As summarized above, the subject invention provides methods for identifying a fluid deposited onto a substrate surface by a fluid deposition device such as a pulse-jet fluid deposition device. In accordance with the subject methods, a fluid is deposited from a fluid deposition device such as a pulse-jet fluid deposition device onto a substrate surface to provide a spot of the fluid on the substrate surface. The deposited spot is evaluated based on one or more physical characteristics of the spot to identify the fluid, where the identity determined may include the type or kind, i.e., the name of the composition, of fluid and/or whether the fluids includes contaminants or impurities which may render the fluid unusable for its intended purpose. It is to be understood that the subject invention is not limited to any particular fluid deposition device and thus may be employed with any type of fluid deposition device such as any type of pulse-jet deposition device, e.g., may be employed with both thermal or piezo pulse jet devices, etc, or other suitable fluid deposition device such as, but not limited to, fluid deposition devices that employ other deposition technologies such as spotting a fluid with a pin or acoustical focusing and the like. Representative fluid deposition devices suitable for use in practicing the subject invention include, but are not limited to, those described in International Patent Application Publication Nos.: WO 95/25116 and WO 98/41531 and U.S. Pat. Nos.: 6,242,266; 6,232,072; 6,180,351; 6,323,043; 6,447,723; 5,028,937; 5,807,522; the disclosures of which are herein incorporated by reference in their entirety, including the references cited therein.

The fluid deposition device may be manual, e.g., a manually operated pipette or fluid reservoir, or may be partially or completely automated. The subject invention will be described primarily with reference to a pulse-jet fluid deposition device for ease of description only, where such description is in no way intended to limit the scope of the invention as it will be apparent that a wide variety of fluid deposition devices may be employed.

Accordingly, the first step of the methods of the subject invention is to deposit a volume of fluid onto a substrate surface to provide a deposited spot of the fluid on the surface of the substrate. The subject methods will be primarily described herein with respect to the deposition and identification of a single fluid, however it is to be understood that a plurality of fluids (where some or all of the fluids may be the same or some or all of the fluids may be different) may be deposited on a substrate surface at different locations (e.g., deposited at the same or different times). In the embodiments wherein a plurality of fluids is deposited onto the same substrate surface, some or all of the fluids may be evaluated at the same time or some or all of the fluids may be evaluated at different times, such that a plurality of fluids may be identified simultaneously or in parallel or sequentially.

It will be apparent upon reading this disclosure that the subject methods may be employed in a wide variety of applications where a fluid is deposited onto a substrate surface. Accordingly, the subject methods may be employed in the identification of a wide variety of fluids. In many embodiments, the subject methods are employed in array synthesis protocols, or rather may be employed to identify one or more fluids for use in an array synthesis protocol or rather one or more fluids that may be used to produce one or more polymers or biopolymers on a substrate surface, as will be described in greater detail below. In further describing the subject invention, fluids of an array synthesis protocol will be primarily referenced in describing the subject methods, where such reference is for exemplary purposes only and is in no way intended to limit the scope of the invention. As briefly described above, the synthesis of biopolymers on a substrate surface in array production involves a number of fluids, where any of these fluids may be employed in the subject invention. Such fluids include, but are not limited to, fluid monomers, e.g., nucleotides or nucleosides or rather deoxynucleoside phosphoramidites such as deoxyadenosine phosphoramidite, deoxyguanosine phosphoramidite, deoxycytidine phosphoramidite, and deoxythrymidine; amino acids, saccharides, peptides; fluid activators, e.g., tetrazole and tetrazole derivatives such as S-ethyl tetrazole, dicyanoimidazole (“DCI”), benzimidazolium triflate, and the like; capping fluids, e.g., a capping solution including acetic anhydride, pyridine or 2,6-lutidine (2,6-dimethylpyridine), and tetrahydrofuran (“THF”), or a capping solution including 1-methyl-imidazole in THF; oxidizing fluids, e.g., an oxidizing solution including iodine in THF, pyridine, and water; deprotecting fluids, e.g., acids; washing fluids; buffering fluids; quality control standards, etc.

Thus, in accordance with the subject methods, a volume of a fluid is deposited at a surface location of a substrate by a fluid deposition device such as a pulse-jet fluid deposition device to provide a spot of the fluid at a specific location on the substrate surface. The volume of fluid expelled from the deposition device, and subsequently deposited on the substrate surface, may vary depending on the particular fluid under investigation, etc., however usually a volume greater than about 20 pL will be employed and in many embodiments a volume greater than about 90 pL will be employed. In certain embodiments, the volume of fluid deposited onto the substrate surface ranges from about 20 pL to about 160 pL or more, usually from about 60 pL to about 140 pL and more usually from about 90 pL to about 120 pL. A fluid may be deposited onto a substrate surface, i.e., evaluated according to the subject methods, at any appropriate time. For example, the subject methods may be employed during an initial set-up or installation of fluids with a fluid deposition device such as at the beginning of a manufacturing shift change or the like, or may be employed subsequent to any change of fluids such as to replace a depleted fluid reservoir during a protocol.

The particulars of a substrate upon which the fluid is deposited is not particularly important to the subject methods as a wide variety of substrates may be employed. One requirement of a substrate is that it does not adversely interfere with the evaluation of one or more physical characteristics of a deposited spot to such an extent that the fluid of the spot is not able to be identified due to the substrate. Furthermore, in those embodiments that evaluate a spot's response to illuminated light such as light absorbency as measured by light reflectance or light transmission as will be described in greater detail below, the substrate will be one that permits such evaluation, e.g., permits measurements of the amount of light transmitted through a spot, etc. In certain embodiments, a substrate may be employed to identify a fluid and the same substrate may be used in a manufacture using the identified fluid, e.g., as an array substrate, or the substrate used to identify a fluid may not be used in a manufacture using the fluid, i.e., another substrate may be used, e.g., another substrate may be used as an array substrate.

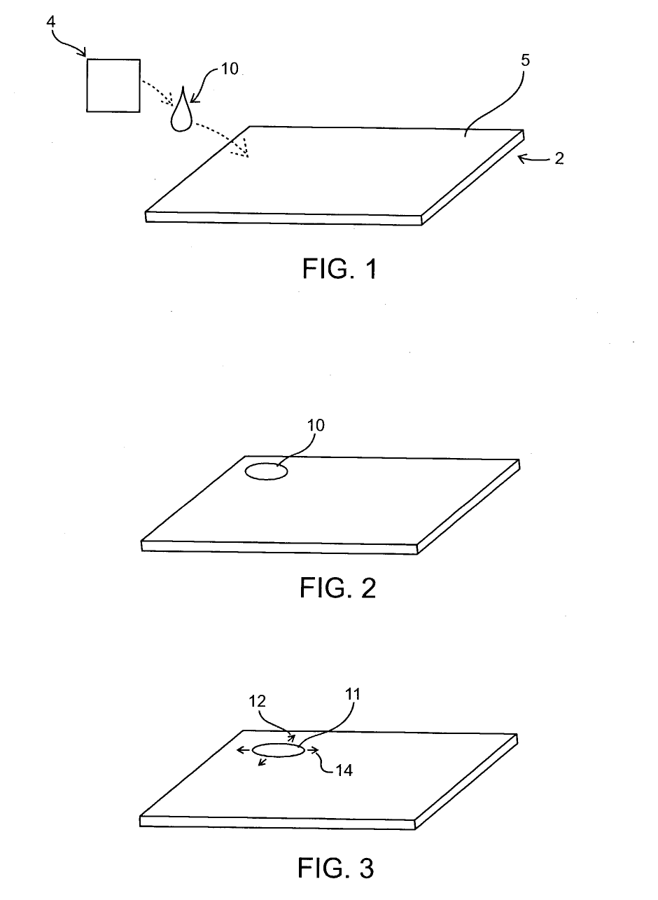

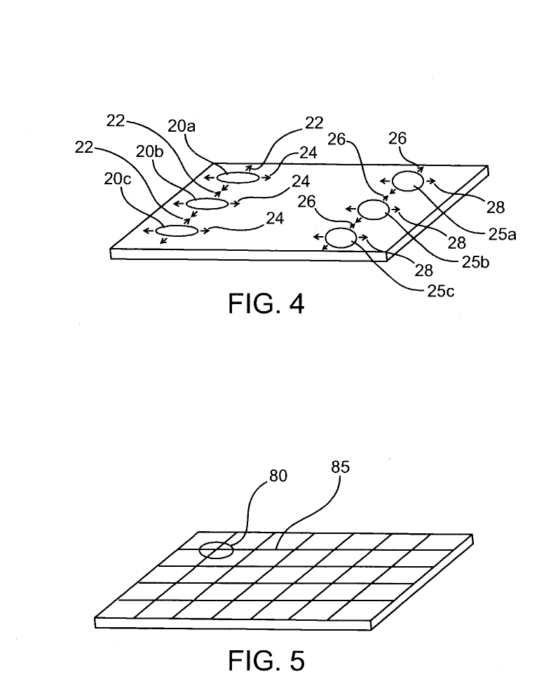

FIGS. 1-5 illustrate the principles of the subject invention. As shown in FIGS. 1 and 2, a volume of a fluid 10 is expelled from a pulse-jet fluid deposition device 4 onto a surface 5 of a substrate 2. As described above, such fluid may be a fluid monomer such as a phosphoramidite monomer, a fluid activator, etc. In many embodiments, replicates of the same reagent may be deposited onto the substrate surface to produce replicates of spots, such as duplicates, triplicates, etc., on a substrate surface (see for example FIG. 4), where each spot may be evaluated in order to determine the identity of the fluid reagent used to produce the spots. In certain embodiments, a volume of a quality control standard and/or a negative control and/or a positive control may be deposited onto a substrate surface, for example in addition to a fluid spot to be identified.

A feature of the subject invention is that the identity of a fluid may be determined by evaluating one or more physical characteristics of a spot present on the substrate surface produced from the fluid. That is, in accordance with the subject methods, unique or different physical characteristics of different fluids are utilized to determine the identity of a fluid. Accordingly, one or more physical characteristics of a spot are evaluated and used to the identity of the fluid. In employing the subject methods, a single physical characteristic of the spot may be evaluated or two or more physical characteristics may be evaluated in order to identify a given fluid.

In general, the physical characteristic(s) which may be evaluated to yield the identification of any given fluid is chosen to be substantially unique to that fluid such that a fluid may be distinguishable from other fluids based upon one or more unique physical characteristics of that fluid. As such, a physical characteristic, or a combination of physical characteristics, may be chosen for evaluation, where the chosen physical characteristic(s) differs amongst the different fluids, i.e., is unique to a particular fluid spot, such that a given fluid may be identified according to the physical characteristic or combination of physical characteristics. For example, in a simple example where the physical characteristic evaluated is spot shape, one or more different reagents that are operatively coupled to, and deposited from, a pulse-jet fluid deposition device may produce unique shapes on the substrate surface, e.g., due to different viscosities, such that a given reagent may be identified according to a given spot's shape.

Accordingly, representative physical characteristics that may be employed to identify a given reagent include, but are not limited to one or more of the following physical characteristics: spot shape, spot size, light absorption, and the like. As described above, any one of these may provide the requisite information to determine the fluid identity or a combination of these may be employed. As noted above, in certain embodiments more than one physical characteristic may be employed.

In certain embodiments the physical characteristic evaluated, or one of the physical characteristics evaluated, is the shape of a deposited spot. For example, fluids with relatively higher viscosities tend to produce spots having shapes that differ from fluids with relatively lower viscosities, e.g., higher viscosity fluids may provide shapes that are more elliptical than lower viscosity fluids. The shape of a spot may be determined in any convenient manner. For example, the shape may be determined by measuring one or more axes of a deposited spot. As such, a ratio of the axes may be determined, e.g., the ratio of a first axis to a second axis, such that the shape is determined by a ratio of the dimensions of the axes. In certain embodiments a first axis may be a minor axis and the second axis may be a major axis. For example, an aspect ratio, represented by the physical length of the vertical axis divided by that of the horizontal axis, may be employed to determine spot shape.

As noted above, fluids having different viscosities may produce spots having different shapes, a comparison of spot shapes may be used to identify a fluid. For example, in certain protocols fluid monomers such as phophoramidite fluids as well as fluid activators such as tetrazole or a tetrazole derivative and the like may be employed. Because phosphoramidite fluids have different viscosities compared to the viscosities of fluid activators. Accordingly, the shape of a spot produced by a phophoramidite fluid will differ from the shape of a spot produced by a fluid activator fluid and thus the identity of the spots produced by these fluids can be easily determined. Accordingly, to identify each fluid or to determine whether each fluid is installed appropriately, i.e., installed at the correct printhead, an evaluation of the shapes of the spots produced by each fluid will enable identification of the fluid that produced the spot because a particular shape may be correlated with a particular fluid, which identification may then enable confirmation or detection of any fluid installation errors.

FIG. 3 illustrates an exemplary embodiment of a spot 11 having a first axis 12 herein shown as a vertical, minor axis and a second axis 14 herein shown as a horizontal, major axis, where the dimensions of the axes of spot 11 may be unique to that fluid spot and thus provide the identity of the fluid. FIG. 4 shows a comparison of two different fluids, each present as multiple spots. Accordingly, a first fluid, e.g., a first fluid monomer, is represented on a substrate surface by three replicate spots 20a, 20b and 20c, each having a first axis 22 and a second axis 24 and a second fluid, e.g., a second fluid monomer, is represented on the substrate surface by three replicate spots 25a, 25b and 25c, each having a first axis 26 and a second axis 28. Of course, additional spots may be deposited also such as deposited spots of fluid activator. Accordingly, each spot provides a unique shape, which shape is determined by the measuring one or more axes of a given spot, e.g., determining a ratio of two of the spot's axes.

Of course, it will be apparent to those of skill in the art that the shape may be determined in a number of different ways such as a determination of the amount and configuration of the surface area of a substrate occupied by the spot, as shown in FIG. 5 which utilizes a grid 85 on the substrate surface to determine the shape of the spot 80 by determining which squares of the grid are contacted by a spot and the amount of surface area of those squares contacted by the spot.

In certain embodiments the physical characteristic evaluated, or one of the physical characteristics evaluated, is the size of the deposited spot. The size of the spot may be determined in any convenient manner. For example, the size of a spot may be determined in manners analogous to those described above for determining the shape of a spot. For example, different fluids may spread about the substrate surface more than others, e.g., due to viscosity, surface tensions, etc., thus providing a unique size or a size that is unique relative to the sizes of one or more other spots. For example, a first fluid may produce a first spot having a particular size, which spot size may be directly correlated with the identity of the fluid. Alternatively, the spot size of the first fluid may be compared with the size of a second spot produced from a second fluid and this relative comparison may provide the identity of the fluids, e.g., if it is known that one of the fluids produces spots that are larger than the spots produced by the other fluid.

In certain embodiments the physical characteristic evaluated, or one of the physical characteristics evaluated, is the light absorbency and/or light scattering of the deposited spot. The light absorbency of the spot may be determined in any convenient manner. Accordingly, in such embodiments a spot is illuminated with light and either the light reflected back from or transmitted through the spot is measured to determine the amount of light absorbed by the spot. In such instances, the wavelength of the light used to evaluate a spot may vary where any suitable wavelength of the spectrum may be employed. In certain embodiments, the wavelength(s) employed may be in the visible/UV range, where in certain embodiments two different wavelengths may be employed to illuminate a spot, or different wavelengths may be employed to illuminate different spots on a substrate.

In certain embodiments the physical characteristic evaluated the centroid position of a spot may be determined. For example, prior to determining one or more other physical characteristics, the centroid position of a spot may be determined in order to provide information about the proper functioning of the fluid deposition device, e.g., to determine any deposition device errors. More specifically, the fluid deposition or spot of each reagent is positioned on a precise or predetermined position of a substrate. If an error in deposition occurs during deposition of a given reagent such that the reagent is unintentionally deposited in a different position on a substrate surface, e.g., the reagent is not centered in the correct location of the substrate surface, the determination of one or more physical characteristics may be compromised. Accordingly, the centroid of each reagent may be determined to verify the correct positioning of the reagent on the substrate surface. The centroid of a reagent may be determined in any convenient manner and may include determining the predetermined or intended location on a substrate surface and comparing that intended location to the actual position of a deposited reagent. If the comparison yields a differential that is greater than a particular threshold or margin of error, the fluid deposition device may be adjusted to correct for the deposition error.

As noted above, the subject methods may also be employed to identify whether any contaminants or impurities are present in a fluid, where such may be determined in manners analogous to those described above. For example, in such instances the type of fluid may be known, e.g., it may be known that the fluid is a tetrazole fluid. One or more physical characteristics of a spot of the fluid having a purity level that is acceptable for its intended use may also be known. Accordingly, an evaluation of one or more of the physical characteristics of a deposited spot of the fluid can provide information about the purity of the fluid. For example, if it is known that a given fluid having a particular purity level produces a particular spot size on a substrate surface, a comparison of the deposited spot size to the known or expected spot size yields information about the purity of the fluid. For example, a certain deviation from the expected spot size may indicate a fluid is contaminated or has impurities which may render the fluid unsuitable for its intended use.

The subject methods also include adjusting a protocol, such as an array synthesis protocol, based on the determined identity of a fluid and/or the suitability of the fluid. Accordingly, when the identity of a fluid is determined by the subject methods, it may be that one or more fluids has been mislabeled, mixed-up or is contaminated. Accordingly, the fluid may be removed from the fluid deposition device and replaced with another fluid, where the subject methods may be reiterated one or more times.

As noted above, evaluating at least one physical characteristic of a deposited spot enables the identity of the fluid employed to produce the spot to be known. In certain embodiments, this is accomplished by providing a data set of at least one physical characteristic of at least one fluid. Usually, such a data set includes a plurality of different physical characteristics, each corresponding to a particular or known fluid. For example, a data set may include different physical characteristics such as spot shape, spot size and spot light absorbency, where each of these physical characteristics, e.g., spot shape, may then include one or more, usually a plurality, of physical characteristics of different fluids, e.g., a plurality of spot shapes, where each correlates to a particular fluid and/or fluid purity. In this manner, the physical characteristic(s) of the deposited spot of interest may be compared to this data set. If a comparison between the physical characteristic(s) of the fluid under investigation and the physical characteristic(s) present in the data set yields a matching physical characteristic(s), the identity of the fluid may be determined by this match. For example, at least one physical characteristic of a deposited spot may be evaluated according to the subject methods. For the sake of this example, spot shape will be used as the physical characteristic, where such is for exemplary purposes only and is in no way intended to limit the subject invention. Accordingly, once the shape of a spot is determined, the shape may be compared to at least one shape included in such a data set to find a matching shape. If a matching shape is found in the data set, the identity of the fluid of interest may be determined. Such a data set may be provided or embodied on a tangible medium such as paper and the like. In certain embodiments, the data set is provided on a computer-readable medium and in certain embodiments the matching protocol is performed by appropriate hardware/software (e.g., a processor) such that some or all of the matching/correlating protocol may be performed automatically, as will be described in greater detail below.

Programming for practicing at least certain embodiments of the subject methods is also provided, as described above. For example, programming may be provided to direct a processor to execute some or all of the steps for practicing the subject methods. For example, in certain embodiments as described above, the methods employ a device that includes a plurality of pulse jets wherein the device is configured to deposit fluid, typically a plurality of fluids, onto a substrate surface, e.g., to produce an array. In such methods, programming may be employed that directs the device or other apparatus to identify one or more of the plurality of fluids, typically prior to employing one or more of the fluids in a synthesis protocol, by producing a spot of the fluid onto a substrate surface and evaluating at least one physical characteristic of the deposited spot to determined the identity of the fluid.

The programming may also include a data set as described above.

As noted above, the programming may be configured to control an apparatus for carrying out some or all of the subject methodologies, where the apparatus directed by the programming may be the pulse-jet fluid deposition device employed to deposit the fluid spots, or may be an apparatus other than the pulse-jet fluid deposition device. In those instances where the apparatus is an apparatus other than the pulse-jet fluid deposition device employed to deposit the fluid spots for identification, the apparatus may be completely separate from the deposition device (but may be operatively coupled to the deposition device in certain embodiments) or may be integral with the deposition device. Accordingly, some or all of the subject methods may be automated.

Programming according to the subject invention may be recorded on computer-readable media, e.g., any medium that can be read and accessed directly or indirectly by a computer. Such media include, but are not limited to, magnetic tape, optical storage such as CD-ROM and DVD, electrical storage media such as RAM and ROM, and the hybrids of these categories such as magnetic/optical storage media. One of skill in the art can readily appreciate how any of the presently known computer readable mediums may be used to provide a manufacture that includes a recording of the present programming/algorithm for carrying out the above-described methodology.

In certain embodiments, the system is further characterized in that it provides a user interface, where the user interface presents to a user the option of selecting amongst a plurality of different functions for evaluating a fluid according to the subject methods, for example choosing amongst one or more different, including multiple different, inputs such as amount of fluid employed, the particular physical characteristic(s) evaluated, and the like. The user interface may also present to a user the option of selecting amongst a plurality of different functions for using or rejecting a fluid identified according to the subject methods, e.g., using or rejecting an identified fluid to produce an array.

Utility

The subject invention finds use in a variety of applications wherein a fluid is in need of identification. As noted above, such applications may include the identification of one or more fluids employed to deposit polymers such as biological polymers, i.e., biopolymers, or other moieties on surfaces of a variety of substrates such as in the fabrication of an array. Accordingly, the subject invention is particularly well-suited in the identification of fluids employed to produce an array using a pulse-jet fluid deposition device.

As described above, a number of fluids are employed in the fabrication of an array, where one or more of these fluids may be identified according to the subject methods as described above. Such fluids include, but are not limited to, fluid monomers, e.g., nucleotides or nucleosides or rather deoxynucleoside phosphoramidites such as deoxyadenosine phosphoramidite, deoxyguanosine phosphoramidite, deoxycytidine phosphoramidite, and deoxythrymidine; amino acids, saccharides, peptides; fluid activators, e.g., tetrazole and tetrazole derivatives such as S-ethyl tetrazole, dicyanoimidazole (“DCI”), benzimidazolium triflate, and the like; capping fluids, e.g., a capping solution including acetic anhydride, pyridine or 2,6-lutidine (2,6-dimethylpyridine), and tetrahydrofuran (“THF”), or a capping solution including 1-methyl-imidazole in THF; oxidizing fluids, e.g., an oxidizing solution including iodine in THF, pyridine, and water; deprotecting fluids, e.g., acids; washing fluids; buffering fluids; quality control standards, positive and negative controls, etc.

Once one or more of the fluids are identified in accordance with the subject invention, the identified fluid(s) may be employed in an array synthesis protocol or, if a fluid identification reveals that one or more fluids needs to be replaced, such replacement may be performed and, if necessary, the subject methods may be repeated one or more times. Once all of the appropriate fluids are operatively associated with a pulse-jet fluid deposition device, the pulse-jet fluid deposition device may be employed to deposit the fluids onto a substrate surface to provide an array.

Accordingly, also provided by the subject invention are novel arrays produced using the subject methods. That is, such arrays include at least one fluid identified according to the subject methods. In many embodiments such arrays include a plurality of fluids identified according to the subject methods, where the number of fluids identified according to the subject methods and employed in the fabrication of an array may range from about 1 to about 15 fluids or more, usually from about 4 to about 6 fluids.

Arrays find use in a variety of applications, including gene expression analysis, drug screening, nucleic acid sequencing, mutation analysis, and the like. These biopolymeric arrays include a plurality of ligands or molecules or probes (i.e., binding agents or members of a binding pair) deposited onto the surface of a substrate in the form of an “array” or pattern.

The subject arrays include at least two distinct polymers that differ by monomeric sequence attached to different and known locations on the substrate surface. Each distinct polymeric sequence of the array is typically present as a composition of multiple copies of the polymer on a substrate surface, e.g., as a spot or feature on the surface of the substrate. The number of distinct polymeric sequences, and hence spots or similar structures, present on the array may vary, where a typical array may contain more than about ten, more than about one hundred, more than about one thousand, more than about ten thousand or even more than about one hundred thousand features in an area of less than about 20 cm² or even less than about 10 cm². For example, features may have widths (that is, diameter, for a round spot) in the range from about 10 μm to about 1.0 cm. In other embodiments, each feature may have a width in the range from about 1.0 μm to about 1.0 mm, usually from about 5.0 μm to about 500 μm and more usually from about 10 μm to about 200 μm. Non-round features may have area ranges equivalent to that of circular features with the foregoing width (diameter) ranges. At least some, or all, of the features are of different compositions (for example, when any repeats of each feature composition are excluded, the remaining features may account for at least about 5%, 10% or 20% of the total number of features). Interfeature areas will typically (but not essentially) be present which do not carry any polynucleotide (or other biopolymer or chemical moiety of a type of which the features are composed). It will be appreciated though, that the interfeature areas, when present, could be of various sizes and configurations. The spots or features of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like.

An array includes any one or two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the arrays are arrays of polymeric or biopolymeric ligands or molecules, i.e., binding agents, where the polymeric binding agents may be any of: peptides, proteins, nucleic acids, polysaccharides, synthetic mimetics of such biopolymeric binding agents, etc. In many embodiments of interest, the arrays are peptide arrays and arrays of nucleic acids, including oligonucleotides, polynucleotides, cDNAs, mRNAs, synthetic mimetics thereof, and the like.

A variety of solid supports or substrates may be used, upon which an array may be positioned. As noted above, the same substrate employed in the identification of at least one fluid may serve as an array substrate such that one or more fluids may be identified according to the subject methods using a substrate and the same substrate may then be used in the production of an array using the one or more identified fluids. In certain embodiments, another substrate, different from the substrate employed to identify one or more fluids, may be used as an array substrate such that one or more fluids may be identified according to the subject methods using a first substrate and a second substrate may then be used in the production of an array using the one or more identified fluids. In certain embodiments, a plurality of arrays may be stably associated with one substrate. For example, a plurality of arrays may be stably associated with one substrate, where the arrays are spatially separated from some or all of the other arrays associated with the substrate.

The same pulse-jet fluid deposition device employed in the fluid identification methods of the subject invention may be employed in the fabrication of an array such that the fluids necessary in the production of an array may be delivered by the pulse-jet fluid deposition device.

The array substrate may be selected from a wide variety of materials including, but not limited to, natural polymeric materials, particularly cellulosic materials and materials derived from cellulose, such as fiber containing papers, e.g., filter paper, chromatographic paper, etc., synthetic or modified naturally occurring polymers, such as nitrocellulose, cellulose acetate, poly (vinyl chloride), polyamides, polyacrylamide, polyacrylate, polymethacrylate, polyesters, polyolefins, polyethylene, polytetrafluoro-ethylene, polypropylene, poly (4-methylbutene), polystyrene, poly(ethylene terephthalate), nylon, poly(vinyl butyrate), cross linked dextran, agarose, etc.; either used by themselves or in conjunction with other materials; fused silica (e.g., glass), bioglass, silicon chips, ceramics, metals, and the like. For example, substrates may include polystyrene, to which short oligophosphodiesters, e.g., oligonucleotides ranging from about 5 to about 50 nucleotides in length, may readily be covalently attached (see for example Letsinger et al. (1975) Nucl. Acids Res. 2:773-786), as well as polyacrylamide (see for example Gait et al. (1982) Nucl. Acids Res. 10:6243-6254), silica (see for exampler Caruthers et al. (1980) Tetrahedron Letters 21:719-722), and controlled-pore glass (see for exampler Sproat et al. (1983) Tetrahedron Letters 24:5771-5774). Additionally, the substrate can be hydrophilic or capable of being rendered hydrophilic.

Suitable substrates may exist, for example, as sheets, tubing, spheres, containers, pads, slices, films, plates, slides, strips, disks, etc. The substrate is usually flat, but may take on alternative surface configurations. The substrate can be a flat glass substrate, such as a conventional microscope glass slide, a cover slip and the like. Common substrates used for the arrays of probes are surface-derivatized glass or silica, or polymer membrane surfaces, for example as described in Maskos, U. et al., Nucleic Acids Res, 1992, 20:1679-84 and Southern, E. M. et al., Nucleic acids Res, 1994, 22:1368-73.

The array substrate surface may be smooth or substantially planar, or have irregularities or surface modifications, such as depressions or elevations. The surface may be modified with one or more different layers of compounds that serve to modify the properties of the surface in a desirable manner. Such modification layers of interest include: inorganic and organic layers such as metals, metal oxides, polymers, small organic molecules and the like and may include functional moieties, such as hydroxyl groups, attached thereto (for example, conjugated).

Each array may cover an area of less than about 100 cm², or even less than about 50 cm², 10 cm² or 1 cm². In many embodiments, the substrate carrying the one or more arrays will be shaped generally as a rectangular solid (although other shapes are possible), having a length of more than about 4 mm and less than about 1 m, usually more than about 4 mm and less than about 600 mm, more usually less than about 400 mm; a width of more than about 4 mm and less than about 1 m, usually less than about 500 mm and more usually less than about 400 mm; and a thickness of more than about 0.01 mm and less than about 5.0 mm, usually more than about 0.1 mm and less than about 2 mm and more usually more than about 0.2 and less than about 1 mm. Substrates having shapes other than rectangular may have analogous dimensions. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least about 20%, or about 50% (or even at least about 70%, 90%, or 95%), of the illuminating light incident on the substrate as may be measured across the entire integrated spectrum of such illuminating light or alternatively at 532 nm or 633 nm.

Utility of Arrays

The arrays produced according to the subject invention find use in a variety applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc. The presence of the analyte in the sample is then deduced from the detection of binding complexes on the substrate surface.

Specific analyte detection applications of interest include, but are not limited to, hybridization assays in which the nucleic acid arrays of the subject invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Palo Alto, Calif. Other suitable apparatuses and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample).

The results of the reading (processed or not) may be forwarded (such as by communication) to a remote location if desired, and received there for further use (such as further processing). In certain embodiments, the subject invention include a step of transmitting data from at least one of the detecting and deriving steps, as described above, to a remote location. By “remote location” is meant a location other than the location at which the array is present and the array assay, e.g., hybridization, occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, Internet, etc.

Kits

Finally, kits for use in practicing the subject invention are also provided.

The subject kits at least include at least a computer readable medium including programming as described above and instructions. Such a computer-readable medium may also include a data set of at least one physical characteristic of at least one fluid, as described above. The instructions may include installation and/or set-up directions. The instructions may include directions for use of the invention. For example, the instructions may include directions for using the computer-readable program to identifying a fluid deposited onto a substrate surface by a pulse jet fluid deposition device.

Providing programming and instructions as a kit may serve a number of purposes. The combination may be provided in connection with an apparatus such as a new pulse-jet fluid deposition device for depositing a fluid on a substrate surface according to the subject invention and/or a new apparatus for evaluating at least one physical characteristic of a fluid deposited on a substrate surface in accordance with the subject invention and/or a new apparatus for fabricating an array, where in certain embodiments at least two of the above described apparatuses are the provided in a single apparatus, i.e., are integrated into the same apparatus or system, as described above. Regardless of whether one or more apparatuses employed to practice any or all of the subject invention are separate components or are integrally or otherwise operatively associated together, in this manner of providing a subject kit the programming may be preloaded on one or more of these apparatuses. In this manner, the instructions will serve as a reference manual (or a part thereof) and the computer readable medium as a backup copy to the preloaded programming.

The instructions may be printed on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

In yet other embodiments, the instructions may not themselves be present in the kit, but means for obtaining the instructions from a remote source, e.g., via the Internet, are provided. An example of this embodiment is a kit that includes a World Wide Web address where the instructions may be viewed and/or from which the instructions may be downloaded. Conversely, means may be provided for obtaining the subject programming from a remote source, such as by providing a World Wide Web address. Still further, the kit may be one in which both the instructions and the programming are obtained or downloaded from a remote source, such as the Internet or World Wide Web. Some form of access security or identification protocol may be used to limit access to those entitled to use the subject invention. As with the instructions, the means for obtaining the instructions and/or programming is generally recorded on a suitable recording medium.

The kit may further include one or more fluids for use in the subject invention, i.e., for deposition onto a substrate surface using a pulse-jet fluid deposition device. The one or more fluids may be fluids employed in the fabrication of an array and/or one or more additional components necessary for carrying out an array assay, e.g., an analyte detection assay, such as sample preparation reagents, buffers, labels, and the like. As such, the kits may include one or more containers such as vials or bottles, with each container containing a separate component. Such fluids include, but are not limited to one or more of: fluid monomers, e.g., nucleotides or nucleosides or rather deoxynucleoside phosphoramidites such as deoxyadenosine phosphoramidite, deoxyguanosine phosphoramidite, deoxycytidine phosphoramidite, and deoxythrymidine; amino acids, saccharides, peptides; fluid activators, e.g., tetrazole and tetrazole derivatives such as S-ethyl tetrazole, dicyanoimidazole (“DCI”), benzimidazolium triflate, and the like; capping fluids, e.g., a capping solution including acetic anhydride, pyridine or 2,6-lutidine (2,6-dimethylpyridine), and tetrahydrofuran (“THF”), or a capping solution including 1-methyl-imidazole in THF; oxidizing fluids, e.g., an oxidizing solution including iodine in THF, pyridine, and water; deprotecting fluids, e.g., acids; washing fluids; buffering fluids; quality control standards, denaturation reagent for denaturing an analyte, buffers for an array assay such as hybridization buffers, enzyme substrates, reagents for generating a labeled target sample such as a labeled target nucleic acid sample, negative and positive controls for an array assay, etc.

### Experimental

The following experiment is put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Four experiments were conducted which identify fluids by evaluating the physical characteristic of the shape of a spot deposited onto a substrate surface from a pulse jet fluid deposition device. Accordingly, six different fluids were deposited onto a substrate surface according to the subject methods. The six fluids were: (1) dA-tBPA phosphoramidite fluid (“A-well”), (2) dG-tBPA phosphoramidite fluid (“G-well”), (3) dC-tBPA phosphoramidite fluid (“C-well”), (4) dT-CE phosphoramidite fluid (“T-well”), (5) S-ethylthio-1H-tetrazole fluid (“T 1-well”), and (6) S-ethylthio-1H-tetrazole fluid (“T2-well”), where tBPA=Tert Butyphenoxyacetyl; CE=Cyanoethyl. A volume of 100-120 pL of each fluid was employed to produce a spot onto a surface of silylated glass.

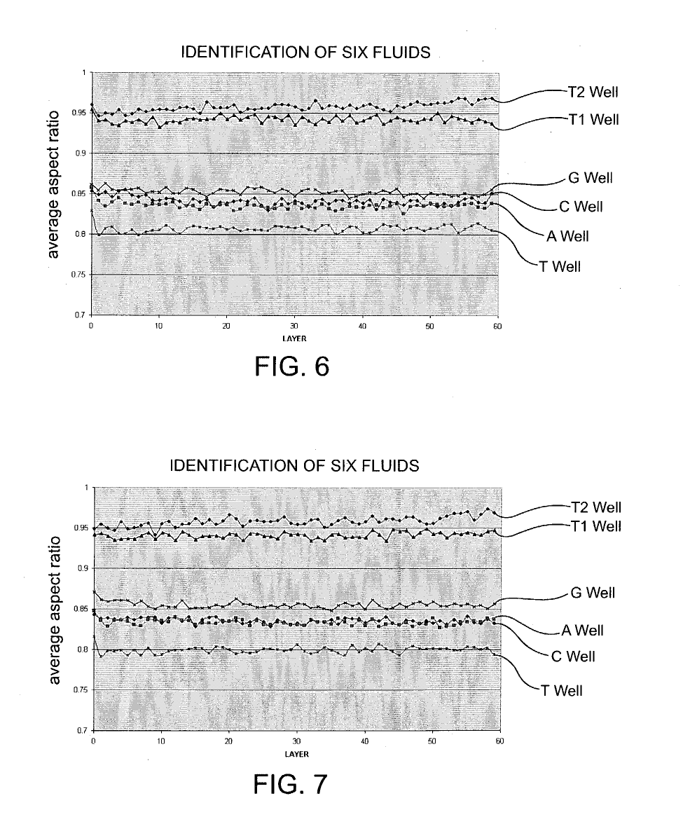

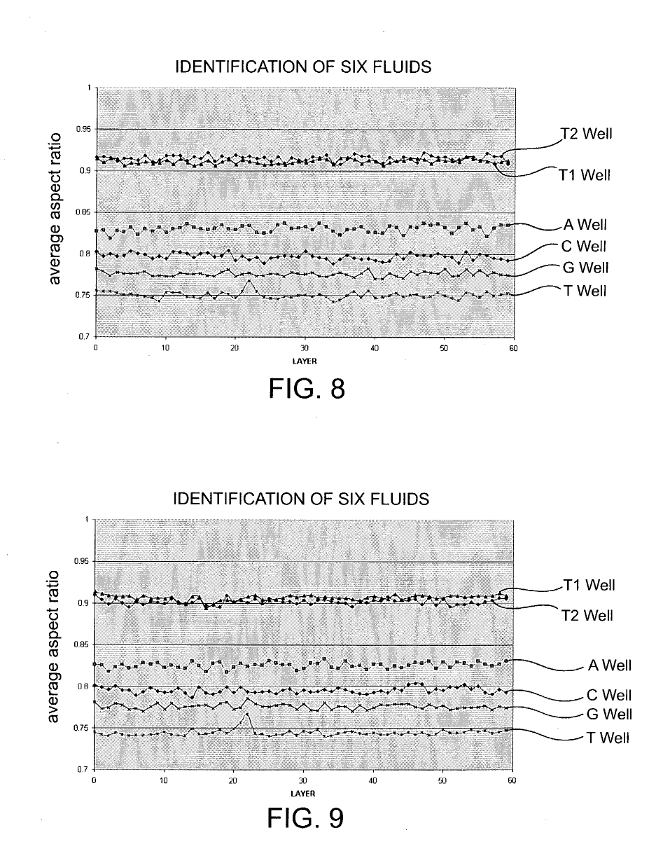

Sixty layers or iterations of each fluid was deposited such that a first spot providing a first layer of a given fluid was deposited on a glass surface and the shape thereof was evaluated by determining the aspect ratio of this first spot. This was repeated fifty-nine more times such that, following the determination of the shape of the first spot, a second spot was deposited at the same location as the first spot thus providing a second layer of the fluid on the glass surface and the shape thereof was evaluated by determining the aspect ratio of the second spot, etc. Twenty different nozzles were employed for each reagent such that a particular printhead of the reagent deposition device employed to deposit a given reagent included twenty nozzles/reagent. In the experiments provided, for any given reagent, all twenty nozzles were employed to deposit the particular reagent at twenty different positions on the substrate surface and the aspect ratio of each of the twenty depositions (at each layer of deposition) was determined. Thus, for a given reagent the twenty aspect ratios were averaged to provide an average aspect ratio (for each layer of deposition). The average aspect ratio was then plotted on a graph (y-axis) versus the layer number (x-axis). Accordingly, the average aspect ratio of the features deposited from a fluid deposition device printhead versus layer was plotted (see FIGS. 6-9). In the examples, the fluid deposition devices employed included two different printheads, where each printhead included three sets of nozzles: a first set of twenty nozzles for deposition of a first monomer, a second set of twenty nozzles for deposition of a second monomer and a third set of twenty nozzles for deposition of tetrazole.

The results show that the identity of each fluid is easily determined by evaluating the shape of a spot produced by depositing a volume of a fluid from a pulse-jet fluid deposition device onto a substrate surface. FIGS. 6-9 show the results of these four examples wherein the average aspect ratio of the features versus layer was plotted. Accordingly, the legend is as follows: the C Well indicates the spots produced from the dC-tBPA phosphoramidite, the T Well indicates the dT-CE phosphoramidite, the A Well indicates the dA-tBPA phosphoramidite, the G Well indicates dG-tBPA phosphoramidite, the T1 Well indicates S-ethylthio-1H-tetrazole and the T2 Well indicates S-ethylthio-1H-tetrazole. As can be seen from the graphs, each fluid produces a unique spot shape, i.e., a spot shape different from the other fluids. Accordingly, the identity of each fluid was easily determined by the spot shape of the fluids.

It is evident from the above results and discussion that the above described invention provides effective methods and devices for identifying a fluid deposited onto a substrate surface by a fluid deposition device such as a pulse-jet fluid deposition device. The subject invention provides for a number of advantages including, but not limited to, ease of use, cost effectiveness, and may be partially or completely automated. Specifically, the subject at least reduces and often eliminates reagent misidentifications or mix-ups, fluid installation errors of a pulse-jet fluid deposition device and use of reagents that are contaminated or have impurities which may render them unsuitable for their intended uses. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of identifying a fluid deposited onto a substrate surface by a fluid deposition device, said method comprising:
(a) depositing said fluid from said fluid deposition device onto said substrate surface to produce a spot of said fluid on said substrate surface; and
(b) evaluating at least one physical characteristic of said deposited spot to identify said deposited fluid.

2. The method of claim 1, wherein said evaluating comprises comparing said at least one physical characteristic to a data set comprising physical characteristics of at least one fluid and identifying said fluid based on said comparison.

3. The method of claim 1, wherein said at least one physical characteristic is at least one of size, shape, and light absorption.

4. The method of claim 1, wherein said method is a method of producing an array.

5. The method of claim 4, wherein said array is produced on said substrate surface.

6. The method of claim 4, wherein said array is produced on another substrate surface.

7. The method of claim 1, wherein said fluid comprises a fluid activator.

8. The method of claim 7, wherein said fluid activator is an acid.

9. The method of claim 8, wherein said weak acid is tetrazole or a tetrazole derivative.

10. The method of claim 7, wherein said fluid comprises a fluid monomer.

11. The method of claim 10, wherein said fluid monomer is a nucleoside.

12. The method of claim 1, wherein said fluid deposition device is a pulse-jet fluid deposition device.

13. A biopolymeric array produced according to the method of claim 1.

14. The biopolymeric array of claim 13, wherein said biopolymeric array is a nucleic acid array.

15. A method of detecting the presence of an analyte in a sample, said method comprising:
(a) contacting (i) a biopolymeric array according to claim 13 comprising a polymeric ligand that specifically binds to said analyte, with (ii) a sample suspected of comprising said analyte under conditions that sufficient for binding of said analyte to a biopolymeric ligand on said array to occur; and
(b) detecting the presence of binding complexes on the surface of said array to detect the presence of said analyte in said sample.

16. The method of claim 15, wherein said method further comprises a data transmission step in which a result from a reading of the array is transmitted from a first location to a second location.

17. The method of claim 16, wherein said second location is a remote location.

18. The method of claim 16, wherein said array is a nucleic acid array.

19. A method of receiving a result transmitted according to claim 16.

20. A computer-readable medium comprising a program for controlling an apparatus to:
(a) measure at least one physical characteristic of a spot deposited on a substrate surface from a fluid deposition device; and
(b) evaluate said measurement to identify said deposited fluid.

21. The computer-readable medium of claim 20, wherein said program further communicates the results of said evaluation to a user.

22. The computer-readable medium of claim 20, wherein said program is operatively associated with a user interface which presents to the user the option of selecting amongst a plurality of different functions for using or rejecting said identified fluid to produce a biopolymeric array.

23. The computer-readable medium of claim 20, wherein said at least one physical characteristic is at least one of size, shape, and light absorption.

24. The computer-readable medium of claim 20, wherein said program further comprises a data set of physical characteristics of at least one fluid, wherein said evaluating comprises comparing said measurement to said data set.

25. The computer-readable medium of claim 20, wherein said apparatus is said pulse-jet deposition device.

26. The computer-readable medium of claim 20, wherein said apparatus is separate from said fluid deposition device.

27. A kit for identifying a fluid deposited onto a substrate surface by a fluid deposition device, said kit comprising:
(a) the computer-readable medium of claim 20; and
(b) instructions for using said computer-readable program to identifying a fluid deposited onto a substrate surface by a fluid deposition device.

28. The kit of claim 27, further comprising a data set of physical characteristics of at least one fluid.

29. The kit of claim 27, further comprising one or more fluids for deposition onto a substrate surface using a fluid deposition device

30. The kit of claim 29, wherein said one or more fluids comprises a fluid monomer.

31. The kit of claim 29, wherein said one or more fluids comprises a fluid activator.

32. The kit of claim 27, wherein said fluid deposition device is a pulse jet fluid deposition device.
